(19) United States
(12) Patent Application Publication
(10) Pub. No.: US 2023/0157683 A1
(43) Pub. Date: May 25, 2023

(54) SUTURING INSTRUMENT

(71) Applicant: Palliare Limited, Galway (IE)

(72) Inventors: John O'DEA, Galway (IE); Hilary Elizabeth Barrett, Limerick (IE)

(73) Assignee: Palliare Limited, Galway (IE)

(21) Appl. No.: 17/991,596

(22) Filed: Nov. 21, 2022

(30) Foreign Application Priority Data
Nov. 22, 2021 (IE) S2021/0195

(51) Int. Cl. A61B 17/04

(52) U.S. Cl. CPC A61B 17/0469; A61B 17/0485; A61B 17/0487

(57) ABSTRACT

A suture instrument comprising an elongated outer sleeve (3) and a cannula (9) terminating in a distal piercing tip (12) is slideable within a cannula bore (7) extending through the outer sleeve (3). A plurality of sutures (15), are located in a suture bore (14) extending longitudinally through the cannula (9). Each suture (15) comprises a suture thread extending between a pair of anchor bars (20), and a cinch clip (24), which is located on a loop (22) of the suture thread (7) formed between the anchor bars (20), and is slideable along the loop (22) for reducing the effective length of the suture thread (17) between the anchor bars (20). The anchor bars (20) and the cinch clips (24) of the sutures (15) are aligned end-to-end sequentially along the suture bore (14) of the cannula (9) with the cinch clip (24) of each suture (15) located between the anchor bars (20) thereof. A pusher rod (35) slideable in the suture bore (14) is operable by a linear actuator for sequentially urging the anchor bars (20) and the cinch clip (24) of each suture (15) from the suture bore (14) through the distal piercing tip (12) of the cannula (9).

### FIELD OF THE INVENTION

The present invention relates to a suturing instrument, for example, a suturing instrument for suturing two portions of a human or animal body together, and in particular, though not limited to a suturing instrument for suturing two portions of a lumen, vessel or organ together within the human or animal body.

### BACKGROUND TO THE INVENTION

In the carrying out of minimally invasive surgery, for example, in the carrying out of surgery using an endoscope or a laparoscope, whereby the endoscope or laparoscope is introduced into the interior of a lumen, vessel, organ or cavity in the human or animal body, and where such a lumen, vessel or organ is to be sutured, in general, it is necessary to suture the lumen, vessel or organ internally within the interior of the lumen, vessel or organ. For example, in the carrying out of a bariatric procedure, such as an endoscopic sleeve gastroplasty procedure on a subject, in which a gastric sleeve is formed passing through the stomach of the subject, an endoscope is entered orally through the oesophagus into the stomach of the subject. A suturing instrument is entered into the stomach of the subject through an instrument bore extending through the endoscope for suturing the opposite walls of the stomach together in order to form the gastric sleeve extending through the stomach of the subject.

Another procedure in which it is necessary to suture a lumen, vessel or organ within the interior of the lumen, vessel or organ is in a procedure for everting a polyp in the colon of a subject in order to facilitate subsequent severing of the everted polyp from the colon wall on the external side thereof. Other procedures which require the suturing of a lumen, vessel or organ interiorly in the lumen, vessel or organ, are procedures which are carried out laparoscopically, whereby a laparoscope is entered into a cavity in the human or animal subject, for example, the abdominal cavity, and the laparoscope is then entered from the abdominal cavity into the lumen, vessel or organ in which the procedure is to be carried out. In such cases, a suturing instrument is entered into the lumen, vessel or organ through an instrument bore in the endoscope or the laparoscope, and suturing of the lumen, vessel or organ is carried out by the suturing instrument internally within the lumen, vessel or organ.

Additionally, in laparoscopic surgery it may be necessary to suture a lumen, vessel or organ externally of the lumen, vessel or organ in a cavity, in which the lumen, vessel or organ is located, for example, a lumen, vessel or organ in the peritoneal cavity. In such cases a laparoscope is entered into the peritoneal cavity and a suturing instrument is then entered through an instrument bore in the laparoscope into the peritoneal cavity.

However, known suturing instruments suffer from various disadvantages. For example, in suturing instruments known heretofore, it is necessary to withdraw the suturing instrument after each suture has been inserted in order to recharge the suturing instrument with the next suture. This is inconvenient, and is also time consuming, which in turn results in unnecessary increase in the time required to carry out the procedure, and in turn the time during which a subject must remain under an anaesthetic.

There is therefore a need for a suturing instrument which addresses this problem.

### SUMMARY OF THE INVENTION

According to the invention there is provided a suturing instrument comprising:
an elongated cannula extending from a proximal end to a distal end and having a suture bore extending longitudinally therethrough for accommodating a plurality of sutures therein, the cannula terminating at its distal end in a distal piercing tip,
the sutures being of the type comprising:
   a suture thread terminating at its opposite ends in respective
   anchor elements,
the suture thread being configured to form
   a loop extending from one anchor element to the other one of the anchor elements, and
   a cinch clip located on the loop of the suture thread and being urgeable along the loop towards the anchor elements for progressively reducing the effective length of the suture thread between the anchor elements,
the suture bore of the cannula being configured for accommodating at least a part of the sutures therein with at least the anchor elements of the sutures being located in the suture bore, and
a pusher element for selectively and sequentially discharging the suture from the suture bore through the distal piercing tip of the cannula, and for selectively and sequentially discharging the anchor elements of each suture through the distal piercing tip of the cannula.

In one embodiment of the invention the pusher element is slideable in the suture bore.

In one embodiment of the invention the pusher element extends into the suture bore from the proximal end of the cannula.

In another embodiment of the invention an urging means is provided for urging the pusher element distally in the suture bore for selectively and sequentially discharging the anchor elements from the suture bore.

In one embodiment of the invention the suture bore is configured for accommodating the anchor elements of each suture aligned with each other, and preferably, for accommodating the anchor elements of the respective sutures aligned with each other, and advantageously, for accommodating the anchor elements aligned with the suture bore.

In another embodiment of the invention the suture bore is configured for accommodating elongated anchor elements of the respective sutures, and preferably, for accommodating the elongated anchor elements aligned longitudinally with each other.

In another embodiment of the invention the suture bore is configured for accommodating the anchor elements of the respective sutures aligned end-to-end with each other.

In another embodiment of the invention the suture bore of the cannula is configured for accommodating the cinch clips of the respective sutures, and preferably, the suture bore is configured for accommodating elongated cinch clips of the respective sutures, and preferably, the suture bore is configured to accommodate the cinch clips of the respective sutures longitudinally aligned with the suture bore, and preferably, longitudinally aligned with each other in the suture bore.

In another embodiment of the invention the suture bore is configured to accommodate the cinch clips aligned with the anchor elements of the respective sutures. Preferably, the suture bore is configured for accommodating the cinch clips of the respective sutures aligned end-to-end with adjacent anchor elements.

In another embodiment of the invention the suture bore is configured to accommodate the cinch clip of each suture located between the anchor elements of the corresponding suture.

In an alternative embodiment of the invention the suture bore is configured for accommodating the cinch clip of each suture located distally relative to the anchor elements of the corresponding suture.

In a further alternative embodiment of the invention the suture bore is configured for accommodating the cinch clip of each suture located proximally relative to the anchor elements of the corresponding suture.

In another embodiment of the invention the pusher element is configured to selectively and sequentially discharge the cinch clip and the anchor elements of each suture, from the suture bore through the distal piercing tip of the cannula.

In another embodiment of the invention an elongated longitudinal slot extends longitudinally along the cannula and through the cannula from an outer side to an inner side of the cannula wall for accommodating the suture threads of the respective sutures therethrough.

In another embodiment of the invention the longitudinal slot extends into the cannula from the distal piercing portion thereof.

In one embodiment of the invention the cannula terminates in a distal piercing portion extending proximally from the distal piercing tip, and preferably, the distal piercing portion is substantially rigid for piercing through tissue.

In another embodiment of the invention the cannula comprises an intermediate portion extending from the distal piercing portion to a proximal portion adjacent the proximal end of the cannula. Preferably, the intermediate portion of the cannula is configured to be flexible and bendable for flexing and bending to accommodate manoeuvring of the suturing instrument through bends and curves through which the suturing instrument must be passed to gain access to the lumen, vessel or organ.

In one embodiment of the invention an interrupted helical slot extends through the peripheral wall of the intermediate portion of the cannula, and the interrupted helical slot extends along the intermediate portion thereof. Preferably, the interrupted helical slot extends the length of the intermediate portion of the cannula from the distal piercing portion to the proximal portion thereof.

In one embodiment of the invention the interrupted helical slot comprises a plurality of slot sections extending partly around the peripheral wall of the intermediate portion of the cannula with interrupted sections of the interrupted helical slot located between adjacent pairs of the slot sections.

In one embodiment of the invention the interrupted helical slot is of pitch lying in the range of 0.8 mm to 1.5 mm, and preferably, the pitch of the interrupted helical slot lies in the range of 1.1 mm to 1.3 mm, and advantageously, the pitch of the interrupted helical slot is approximately 1.2 mm.

In another embodiment of the invention the kerf width of the helical slot is approximately 0.025 mm.

In one embodiment of the invention each slot section extends partly around the intermediate portion of the cannula an angular distance in the range of 1700 to 350°. Preferably, each slot section extends partly around the intermediate portion of the cannula an angular distance in the range of 2100 to 290°.

Advantageously, each slot section extends partly around the intermediate portion of the cannula an angular distance in the range of 2200 to 260°. Preferably, each slot section extends partly around the intermediate portion of the cannula an angular distance of approximately 240°.

In another embodiment of the invention each interrupted section of the interrupted helical slot extends partly around the intermediate portion of the cannula an angular distance in the range of 100 to 190°. Preferably, each interrupted section of the interrupted helical slot extends partly around the intermediate portion of the cannula an angular distance in the range of 20° to 130°. Advantageously, each interrupted section of the interrupted helical slot extends partly around the intermediate portion of the cannula an angular distance in the range of 30° to 70°, and ideally, each interrupted section of the interrupted helical slot extends partly around the intermediate portion of the cannula an angular distance of approximately 40°.

In another embodiment of the invention the outer diameter of the cannula lies in the range of 0.7 mm to 1.8 mm, and preferably, the outer diameter of the cannula is approximately 1.27 mm.

In one embodiment of the invention the wall thickness of the cannula lies in the range of 0.05 mm to 0.3 mm, and preferably, the wall thickness of the cannula is approximately 0.0762 mm.

In another embodiment of the invention the suture accommodating bore of the cannula is of transverse cross-section just slightly greater than the maximum transverse cross-sectional dimension of the anchor elements or the cinch clip, whichever of which is of the greatest maximum transverse cross-section.

In another embodiment of the invention the structure bore of the cannula is of transverse cross-section to slideably accommodate the sutures therein with the suture thread extending along the side of each anchor element, and preferably, the suture bore of the cannula is of transverse cross-section to slideably accommodate the sutures with the suture thread extending along the side of the cinch clips.

In one embodiment of the invention a retaining means is provided for releasably retaining the sutures in the suture bore, and preferably, the retaining means is located adjacent the distal end of the cannula, and advantageously, the retaining means is located just slightly spaced apart longitudinally from the distal piercing tip.

In one embodiment of the invention the retaining means comprises a retaining projection extending inwardly into the suture bore, preferably the retaining projection is formed by inwardly impressing a dimple into the cannula wall from the outer surface thereof, and preferably, a portion of the cannula adjacent which the dimple is impressed into the wall of the cannula is configured to be resilient to permit outward movement of the portion of the cannula from which the retaining projection projects, and in turn outward movement of the retaining projection, in order to accommodate passage of the distal most one of the anchor elements or the cinch clip of the distal most suture in the suture bore past the retaining projection in response to distal urging thereof by the pusher element. Preferably, the resilience of the portion of the cannula from which the retaining projection projects is such as to return it to its undisturbed state for in turn returning the retaining projection to engage and retain captive the next distal most one of the anchor elements or the cinch clip of the distal most suture in the suture bore.

In another embodiment of the invention the urging means is configured for urging the anchor elements and the cinch clips of the respective sutures from the suture bore through the distal piercing tip of the cannula.

Preferably, the urging means is configured to urge the pusher element distally selectively and sequentially through a plurality of respective predefined distances for selectively and sequentially discharging the ones of the anchor elements and the cinch clip of each suture in the suture bore of the cannula through the distal piercing tip of the cannula.

In one embodiment of the invention the distance through which the urging means is configured to urge the pusher element distally in the suture bore for discharging each one of the anchor elements and/or each one of the cinch clips corresponds to the length of the distal most one of the anchor elements and/or the cinch clip of the distal most suture in the suture bore, and advantageously, the distance through which the urging means is configured to urge the pusher element distally in the suture bore for discharging each one of the anchor elements and/or each one of the cinch clips is substantially equal to the length of the distal most one of the anchor elements and/or the cinch clip of the distal most suture in the suture bore of the cannula.

In another embodiment of the invention the urging means is configured to urge the pusher element distally in the suture bore for discharging each one of the anchor elements and/or the cinch clips from the suture bore respective first, second and third predefined distances substantially equal to the respective lengths of the anchor elements and the length of the cinch clip.

In one embodiment of the invention the first and second predefined distance are of similar length, and preferably, are of equal length, and advantageously, the third predefined distance is of a substantially similar length to the first predefined distance, and advantageously, the third predefined distance is of length substantially equal to the first predefined distance.

In one embodiment of the invention the urging means comprises a linear motor, and in another embodiment of the invention the urging means comprises a rotary stepper motor.

In one embodiment of the invention a transmission means is provided for transmitting drive from the urging means to the pusher element.

In a further embodiment of the invention a controller is provided for controlling the operation of the urging means, and preferably, the controller is responsive to an activation signal for operating the urging means to urge the pusher element distally in the suture bore through each one of the predefined distances.

Preferably, the controller is responsive to the activation signal for operating the urging means to urge the pusher element distally in the suture bore through the first predefined distance for urging a first one of the anchor elements of the sutures from the suture bore of the cannula through the distal piercing tip thereof.

In another embodiment of the invention the controller is responsive to the activation signal for operating the urging means to urge the pusher element distally in the suture bore through the second predefined distance for discharging a second one of the anchor elements of the sutures from the suture bore of the cannula through the distal piercing tip thereof.

In a further embodiment of the invention the controller is responsive to the activation signal for urging the pusher element distally in the suture bore through the third predefined distance for discharging the cinch clip of the sutures from the suture bore of the cannula through the distal piercing tip thereof.

In one embodiment of the invention the activation signal is derived from a human operable switch.

In one embodiment of the invention the human operable switch comprises a foot operable switch, and preferably, an electrical switch operable by a foot pedal.

In another embodiment of the invention the human operable switch comprises a manually operable switch.

In one embodiment of the invention the controller is programmed to operate the urging means to urge the distal most pusher element distally in the suture bore one of a first predefined distance of the predefined distances to discharge one of the anchor elements adjacent the distal piercing tip from the suture bore through the distal piercing tip.

In another embodiment of the invention the controller is programmed to operate the urging means to urge the pusher element distally in the suture bore one of a second predefined distance of the predefined distances to discharge one of the cinch clips adjacent the distal piercing tip from the suture bore through the distal piercing tip.

In one embodiment of the invention the anchor elements of the respective sutures are of the same length.

In another embodiment of the invention the cinch clips of the respective sutures are of the same length.

In a further embodiment of the invention the cinch clips of the respective sutures are of the same length as the anchor elements thereof.

In one embodiment of the invention the second predefined distance is equal to the first predefined distance, and in another embodiment of the invention the third predefined distance is equal to the first predefined distance.

In another embodiment of the invention the controller is responsive to each activation signal for operating the urging means to urge the pusher element distally through one of the predefined distances.

In one embodiment of the invention the controller comprises a signal processor, and preferably, a programmable signal processor, and advantageously, the signal processor comprises a microcontroller.

In one embodiment of the invention the signal processor is programmed to count the anchor elements and/or the cinch clips, as the case may be, as the anchor elements and the cinch clips are being discharged from the suture bore, in order that the signal processor can determine the identity of the distal most one of the anchor elements and/or the cinch clip in the suture bore of the cannula.

In another embodiment of the invention a sensor is provided for detecting the distal most one of the anchor elements and the cinch clip of the distal most suture in the suture bore, the sensor being configured to produce a signal indicative of the one of the anchor element and the cinch clip which is distal most in the suture bore.

In one embodiment of the invention the sensor is located adjacent the distal end of the cannula, and preferably, the sensor is located in or on the cannula slightly spaced apart longitudinally from the distal piercing tip thereof.

In another embodiment of the invention the suturing instrument comprises an outer sleeve extending from a proximal end to a distal end and having a cannula bore configured for accommodating the cannula therein.

In one embodiment of the invention the cannula is slideable within the cannula bore.

In another embodiment of the invention the cannula is of a length greater than the length of the outer sleeve.

In an alternative embodiment of the invention the outer sleeve is of a length greater than the length of the cannula.

In one embodiment of the invention the outer sleeve terminates adjacent the proximal end thereof in a primary handle, and advantageously, the cannula terminates adjacent the proximal end thereof in a secondary handle. Preferably, the secondary handle is slideable relative to the primary handle for urging the cannula distally and proximally for urging the cannula distally through the distal end of the outer sleeve for urging the distal piercing tip of the cannula, and for withdrawing the distal piercing tip of the cannula into the outer sleeve through the distal end thereof.

The invention also provides the suturing instrument according to the invention comprising a plurality of the sutures located in the suture bore.

In one embodiment of the invention each suture comprises a suture thread terminating at its opposite ends in respective anchor elements, the suture thread being configured to form a loop extending from one anchor element to the other one of the anchor elements, and a cinch clip located on the loop of the suture thread and being urgeable along the loop towards the anchor elements for progressively reducing the effective length of the suture thread between the anchor elements.

In one embodiment of the invention the sutures are located in the suture bore with the anchor elements of each suture located in the suture bore and aligned with each other, and preferably, with the anchor elements of the respective sutures aligned with each other.

In another embodiment of the invention the anchor elements of the respective sutures comprise elongated anchor elements, and preferably, the sutures are located in the suture bore with the elongated anchor elements aligned longitudinally with each other.

In another embodiment of the invention the sutures are located in the suture bore with the anchor elements of the respective sutures aligned with each other end-to-end.

In another embodiment of the invention the sutures are located in the suture bore with the cinch clips of the respective sutures located in the suture bore.

In one embodiment of the invention the cinch clip of each suture comprises an elongated cinch clip, and preferably, the sutures are located in the suture bore with the cinch clips of the respective sutures longitudinally aligned with the suture bore, and preferably, longitudinally aligned with each other in the suture bore.

In another embodiment of the invention the sutures are located in the suture bore with the cinch clips aligned with the anchor elements of the respective sutures. Preferably, the sutures are located in the suture bore with the cinch clips of the respective sutures aligned end-to-end with adjacent anchor elements.

In another embodiment of the invention the sutures are located in the suture bore with the cinch clip of each suture located between the anchor elements of the corresponding suture.

In an alternative embodiment of the invention the sutures are located in the suture bore with the cinch clip of each suture located distally relative to the anchor elements of the corresponding suture.

In a further alternative embodiment of the invention the sutures are located in the suture bore with the cinch clip of each suture located proximally relative to the anchor elements of the corresponding suture.

In one embodiment of the invention the anchor elements of the respective sutures are of the same length.

In another embodiment of the invention the cinch clips of the respective sutures are of the same length.

In a further embodiment of the invention the cinch clips of the respective sutures are of the same length as the anchor elements thereof.

The invention also provides the suturing instrument comprising a plurality of the sutures located in the suture bore.

Additionally, the invention provides a method for suturing two portions of a lumen, vessel or organ in a cavity of the body of a human or animal subject either internally in the interior of the lumen, vessel, organ or cavity, or externally of the lumen, vessel or organ in the interior of the cavity, the method comprising:
entering the suturing instrument according to the invention charged with a plurality of sutures into the one of the lumen, vessel, organ or cavity in which the suturing is to be carried out,
sequentially inserting the sutures in parts of the portions of the lumen, vessel, organ or cavity to be sutured at spaced apart intervals along the respective portions with the suture thread of each suture extending from the anchor elements thereof through the corresponding parts of a pair thereof of the respective portions of the lumen, vessel or organ, with the parts of each pair thereof being located on the respective portions of the lumen, vessel, organ or cavity, and
tightening the sutures to draw the portions of the lumen, vessel or organ together.

In one embodiment of the invention the parts of the portions of the lumen, vessel, organ or cavity are invaginated, and the sutures are inserted in the invaginated parts with the suture thread of each suture extending from the anchor elements thereof through the corresponding pair of the invaginated parts.

In one embodiment of the invention each suture is inserted into the corresponding pair of the invaginated parts by the suturing instrument by sequentially urging the distal piercing tip of the cannula through the invaginated parts of the pair thereof,
each time the cannula has been urged through the respective invaginated parts of one of the pair thereof, the pusher element of the suturing instrument is urged distally in the suture bore of the cannula to discharge the distal most one of the anchor elements of the distal most suture in the suture bore of the cannula through the distal piercing tip of the cannula to deposit that distal most one of the anchor elements on a distal side of the corresponding one of the pair of the invaginated parts, and
withdrawing the cannula from the corresponding one of the invaginated part of the pair thereof with the suture thread extending from the corresponding one of the anchor elements through the corresponding one of the invaginated part of the pair thereof.

In another embodiment of the invention when each of the anchor elements of the suture just discharged from the suture bore are located on the distal sides of the invaginated parts of the pair thereof with the suture thread of the suture extending from the respective anchor elements through the corresponding invaginated parts of the pair thereof, and with the suture thread forming a loop extending through the cinch clip of the suture, the suture is tightened by urging the cinch clip along the loop towards the anchor elements to reduce the effective length of the suture thread extending between the anchor elements.

In another embodiment of the invention the cinch clip of the distal most suture in the suture bore of the cannula of the suturing instrument is discharged from the suture bore through the distal piercing tip of the cannula in the sequence in which the cinch clip of the distal most suture is located with the anchor elements of the distal most suture in the suture bore. Preferably, the cinch clip is urged from the suture bore by urging the pusher element distally in the suture bore.

Preferably, the pusher element is urged distally in the cannula bore through the predefined distance corresponding to the length of the cinch clip for urging the cinch clip from the suture bore.

In another embodiment of the invention the distal piercing tip of the cannula is sequentially urged through respective parts of a wall of the lumen, vessel or organ adjacent the portions to be sutured together, and the pusher element is operated to urge the distal most one anchor elements of the distal most suture in the suture bore of the cannula through the distal piercing tip thereof on each part of the pair thereof of the wall of the lumen, vessel or organ being pierced by the cannula to deposit the corresponding anchor element on the distal side of the wall of the lumen, vessel or organ, and the cannula is withdrawn from the corresponding part of the wall of the lumen, vessel or organ with the suture thread extending from the anchor element through the wall of the lumen, vessel or organ.

In another embodiment of the invention when each of the anchor elements of the suture just discharged from the suture bore of the cannula are located on the distal sides of the wall adjacent the portions thereof to be sutured together, with the suture thread extending from the respective anchor elements through the portions of the wall to be sutured together, and with the suture thread forming the loop extending through the cinch clip, the suture is tightened by urging the cinch clip along the loop of the suture thread thereof towards the anchor elements for reducing the effective length of the suture thread extending between the anchor elements.

In one embodiment of the invention tightening of the sutures commences when the sutures have been inserted in some or all of the pairs of the parts of the wall or through the wall adjacent the portions of the lumen, vessel or organ to be sutured together, and preferably, the sutures are tightened when all of the sutures have been inserted into the respective pairs of the parts or through the wall adjacent the portions of the lumen, vessel or organ.

Preferably, the sutures are sequentially tightened.

In an alternative embodiment of the invention the sutures are simultaneously tightened.

In a further alternative embodiment of the invention each suture is tightened when the suture has been inserted into the corresponding pair of the parts or through the wall adjacent the portions of the lumen, vessel or organ to be sutured together.

In one embodiment of the invention the sutures are tightened by a tightening instrument comprising an engagement means or a grabbing means for engaging or grabbing the loop of each suture, urging the loop engaged or grabbed by the engagement means or the grabbing means to urge the cinch clip of the suture against a distal abutment face of an abutment member of the tightening instrument, and urging the loop through the cinch clip to urge the cinch clip along the loop towards the anchor elements of the suture.

In another embodiment of the invention the suturing instrument comprises the tightening instrument.

In an alternative embodiment of the invention the tightening instrument comprises a separate instrument to that of the suturing instrument.

In one embodiment of the invention the suturing is carried out internally in the interior of the lumen, vessel, organ or cavity, and the suturing instrument is entered into the interior of the lumen, vessel, organ or cavity, and preferably, the invaginated parts extend internally into the interior of the lumen, vessel, organ or cavity.

In an alternative embodiment of the invention the suturing is carried out externally of the lumen, vessel or organ internally in the cavity in which the lumen, vessel or organ is located, and the suturing instrument is entered into the cavity, and preferably, the invaginated parts extend from the lumen, vessel or organ into the cavity.

In another embodiment of the invention each invaginated part of each pair thereof is retained invaginated by a band extending tightly around the invaginated part which preferably, extends around the invaginated part adjacent the root thereof.

In another embodiment of the invention the parts are invaginated by an invaginating instrument, which preferably, sucks the parts to be invaginated into a suction recess thereof, and preferably, each invaginated part is retained in the suction recess while the band is being placed on the invaginated part.

In another embodiment of the invention the invaginating instrument comprises a multiband ligator.

In a further embodiment of the invention the suturing instrument is entered into the lumen, vessel, organ or cavity by an access device, and preferably, the suturing instrument is entered into the lumen, vessel, organ or cavity through an instrument bore in the access device.

Alternatively, the suturing instrument is entered into the lumen, vessel, organ or cavity with the suturing instrument extending longitudinally along an outer side of the access device.

In one embodiment of the invention the suturing instrument is secured to the outer side of the access device, and preferably, is releasably secured to the outer side of the access device.

In another embodiment of the invention the suturing instrument is secured to the outer side of the access device by a plurality of clips spaced apart longitudinally along the suturing instrument and the access device.

In another embodiment of the invention the clips are configured to releasably engage the suturing instrument, and in another embodiment of the invention the clips are configured to releasably engage the access device. Advantageously, the clips are configured to releasably engage both the suturing instrument and the access device.

In another embodiment of the invention the invaginating instrument is entered into the lumen, vessel, organ or cavity through the access device.

In another embodiment of the invention the tightening instrument is entered into the lumen, vessel, organ or cavity through the access device.

In one embodiment of the invention the access device comprises an endoscope, and in another embodiment of the invention the access device comprises a laparoscope.

In a further embodiment of the invention the organ in which the portions thereof are to be sutured together, comprises a stomach, and preferably, the front and side walls of the stomach are to be sutured together.

In one embodiment of the invention the suturing of the stomach is carried out internally in the interior of the stomach, and preferably, the invaginated parts of the stomach extend from the front and rear walls thereof internally into the interior of the stomach.

In one embodiment of the invention the suturing of the front and rear walls of the stomach together is carried out to form a gastric sleeve in a sleeve gastroplasty procedure.

Preferably, the sleeve gastroplasty procedure comprises an endoscopic sleeve gastroplasty procedure.

### Advantages of the Invention

The advantages of the suturing instrument according to the invention are many. A particularly important advantage of the invention is that the suturing instrument according to the invention enables a plurality of sutures to be inserted in a lumen, vessel or organ, without having to withdraw the suturing instrument to recharge the suturing instrument with a new suture, each time a suture has been inserted in the lumen, vessel or organ. This provides the particularly important advantage in that the time required for suturing within a lumen, vessel or organ with a plurality of sutures is significantly reduced. This advantage is achieved by virtue of the fact that the cannula of the suturing instrument defines an elongated suture accommodating bore, in which a plurality of sutures are stored, and a further advantage of the invention is achieved by the provision of the urging means for urging the pusher element, for in turn selectively and sequentially discharging the sutures from the suture bore, and additionally, for urging the cinch clips of the respective sutures from the suture bore.

The provision of the controller for controlling the operation of the urging means for selectively and sequentially urging the sutures from the suture bore, and also for selectively and sequentially urging the anchor elements and the cinch clips of the respective sutures from the suture bore, provides a further advantage, in that the anchor elements and the cinch clips of sutures can be automatically discharged from the suture bore of the cannula by applying an activation signal to the controller.

The provision of the controller as a programmable controller enables selective and sequential discharge of the anchor elements and the cinch clips of the respective sutures from the suture bore in cases where the anchor elements of each suture are of different lengths to each other, and additionally, where the cinch clips are of different length to either of the lengths of the anchor elements. Indeed, the controller may be programmed to discharge anchor elements and cinch clips of sutures in which the anchor elements and the cinch clips of the respective sutures are of different lengths from one suture to the next.

The invention will be more clearly understood from the following description of some preferred embodiments thereof which are given by way of example only with reference to the accompanying drawings, in which:

### BRIEF DESCRIPTION OF THE DRAWINGS

FIG. 1 is a cross-sectional side elevational view of a suturing instrument according to the invention illustrating a part of the suturing instrument in block representation,

FIG. 2 is a cross-sectional side elevational view of a portion of the suturing instrument of FIG. 1 illustrated in a different state to that of FIG. 1,

FIG. 3 is a perspective view of a suture for use in the suturing instrument of FIG. 1,

FIG. 4 is a perspective view of a portion of the suturing instrument of FIG. 1,

FIG. 5 is an enlarged side elevational view of a detail of the portion of FIG. 4 of the suturing instrument of FIG. 1,

FIG. 6 is an enlarged perspective view of another part of the portion of FIG. 4 of the suturing instrument of FIG. 1,

FIG. 7 is a cross-sectional angular end elevational view on the line VII-VII of FIG. 5,

FIGS. 8 to 11 are perspective views of a portion of the suturing instrument of FIG. 1 illustrated in use suturing parts of a stomach together,

FIG. 12 is a perspective view of a greater portion of the stomach of FIGS. 8 to 11 during the suturing procedure illustrated in FIGS. 8 to 11,

FIG. 13 is a cross-sectional side elevational view through the stomach of FIG. 12,

FIG. 14 is a perspective view of the stomach illustrated in FIG. 12 with the suturing procedure completed,

FIG. 15 is a transverse cross-sectional end elevational view of the stomach of FIG. 12 with the suturing procedure completed,

FIG. 16 is a cross-sectional side elevational view of a portion of a suturing instrument according to another embodiment of the invention,

FIG. 17 is a cross-sectional side elevational view of the portion of the suturing instrument of FIG. 16 illustrating the suturing instrument of FIG. 16 in a different state to that of FIG. 16,

FIG. 18 is a perspective view of a portion of a suturing instrument according to a further embodiment of the invention, and

FIG. 19 is a perspective view of a portion of the suturing instrument of FIG. 18.

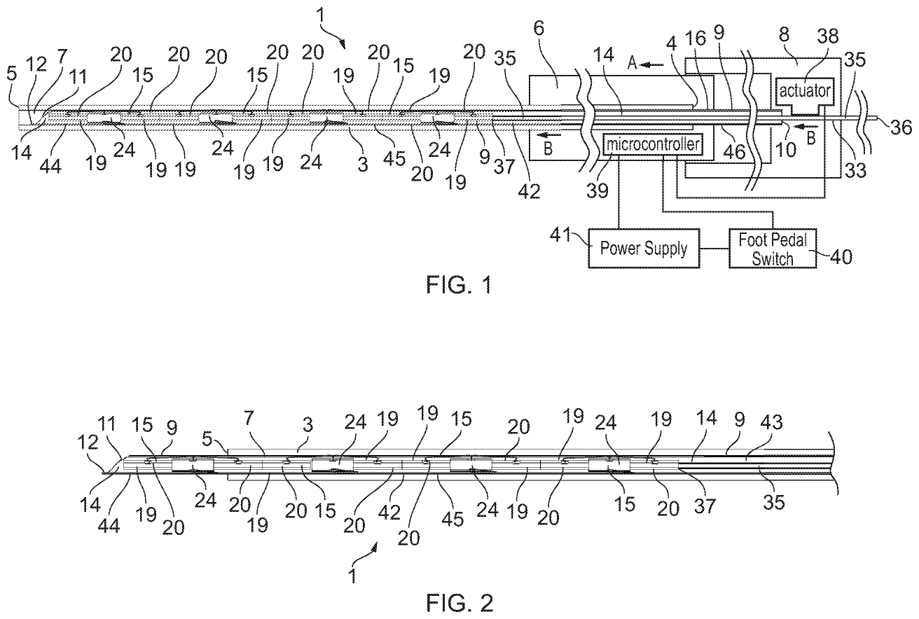

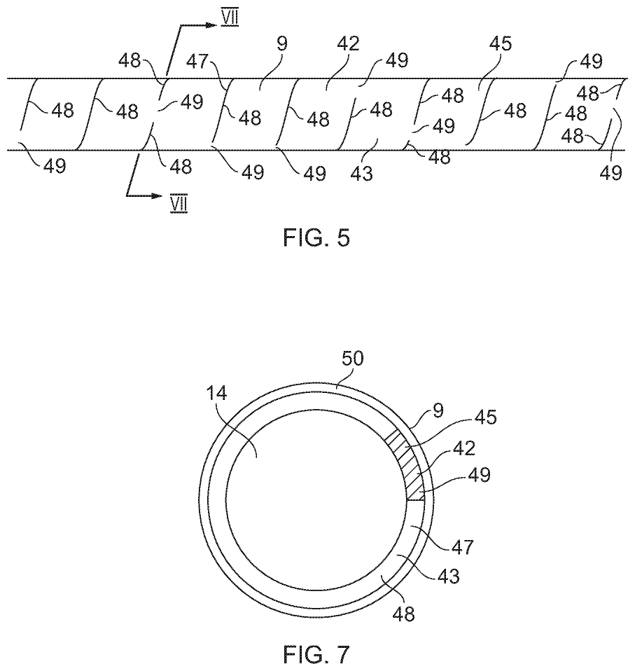

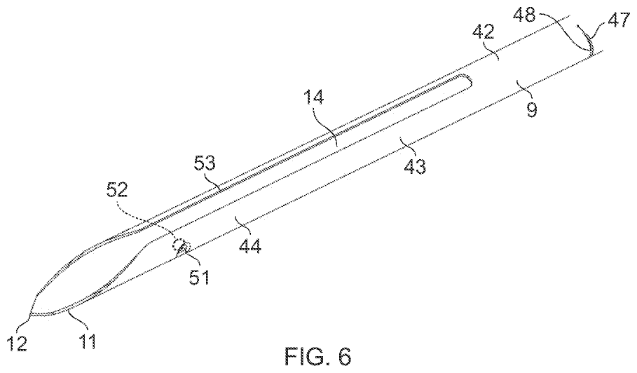

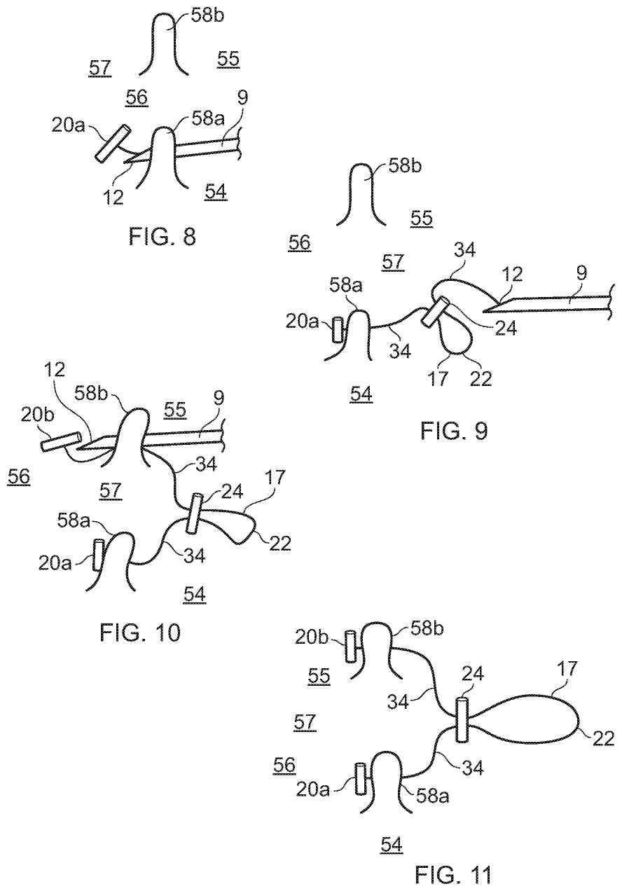

Referring to the drawings and initially to FIGS. 1 to 7 thereof, there is illustrated a suturing instrument according to the invention indicated generally by the reference numeral 1. The suturing instrument 1 is particularly suitable for suturing two portions of a lumen, vessel or organ together with the suturing instrument 1 located internally within the interior of the lumen, vessel or organ, and the suturing of the portions of the lumen, vessel or organ to be sutured together are to be sutured from within the lumen, vessel or organ of a human or animal subject, as will be described below with reference to FIGS. 8 to 15. The suturing instrument 1 is also suitable for suturing a lumen, vessel or organ, where the suturing is being carried out externally of the lumen, vessel or organ in a cavity, for example, in the peritoneal cavity of a human or animal subject.

The suturing instrument 1 comprises an elongated outer sleeve 3 of circular transverse cross-section, extending between a proximal end 4 and a distal end 5. An elongated cannula accommodating bore 7 of circular transverse cross-section extends longitudinally through the outer sleeve 3 from the proximal end 4 to the distal end 5 for slideably accommodating an elongated cannula 9 extending longitudinally therein. The outer sleeve 3 terminates adjacent the proximal end 4 thereof in a primary handle 6 to which the outer sleeve 3 is rigidly secured.

The cannula 9 extends from a proximal end 10 to a distal end 11, and terminates in its distal end 11 in a distal piercing tip 12. The cannula 9 is of circular transverse cross-section and is slideable in the cannula bore 7 of the outer sleeve 3. An elongated suture accommodating bore 14 of circular transverse cross-section extends longitudinally through the cannula 9 from the proximal end 10 to the distal end 11 for accommodating a plurality of sutures 15 therein as will be described below.

The cannula 9 is slideable in the outer sleeve 3 from a withdrawn state illustrated in FIG. 1 to an extended state illustrated in FIG. 2. In the withdrawn state of the cannula 9, the distal piercing tip 12 is located within the distal end 5 of the outer sleeve 3 to protect the distal piercing tip 12, and also to prevent inadvertent piercing of tissue of the lumen, vessel or organ by the distal piercing tip 12. In the extended state of the cannula 9, the distal piercing tip 12 extends outwardly from the distal end 5 of the outer sleeve 3 for piercing tissue into which one of the sutures 15 are to be inserted during suturing of the lumen, vessel or organ.

A secondary handle 8 is slideably mounted on the primary handle 6, and is rigidly connected to the cannula 9 adjacent the proximal end 10 for operating the cannula 9 in the cannula bore 7 of the outer sleeve 3 between the withdrawn state and the extended state. The cannula 9 extends from the proximal end 4 of the outer sleeve 3 to the secondary handle 8 through a bore 16 extending through the primary handle 6. By urging the secondary handle 8 distally in the direction of the arrow A, relative to the primary handle 6, the cannula 9 is urged from the withdrawn state within the outer sleeve 3 to the extended state with the distal piercing tip 12 extending distally outwardly from the distal end 5 of the outer sleeve 3.

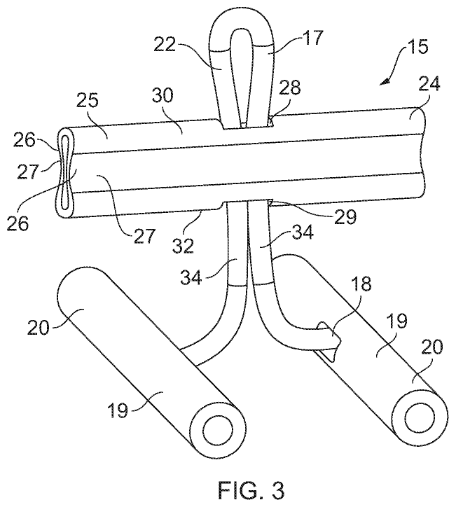

Before describing the suturing instrument 1 in further detail, one of the sutures 15 will first be described with reference to FIG. 3. The sutures 15 are identical to each other. Each suture 15 comprises a suture thread 17 terminating in its opposite ends 18 in respective anchor elements 19. The anchor elements 19 of each suture 15 comprise respective tubular anchor bars 20 of external circular, substantially constant transverse cross-section of diameter which is less than the diameter of the suture bore 14 of the cannula 9, so that the anchor bars 20 are slideable within the suture bore 14. The anchor bars 20 of the anchor elements 19 of the respective sutures 15 are of the same diameter and of the same length. The anchor bars 20 anchor the suture 15 in the respective parts of the lumen, vessel or organ to be sutured together as will be described below. The suture thread 17 is secured in each anchor bar 20 intermediate the respective ends of the anchor bar 20, and extends from the anchor bars 20 to form a loop 22.

The loop 22 of each suture 15 extends through a corresponding cinch clip 24 which is formed from a tubular member 25, opposite side walls 26 of which are crimped towards each other at 27. Top and bottom slots 28 and 29 formed in top and bottom walls 30 and 32, respectively, of the cinch clip 24 accommodate the loop 22 of the suture thread 17 therethrough. The crimping 27 of the side walls 26 of the tubular member 25 is such as to form a friction grip on legs 34 of the loop 22. The friction grip exerted on the legs 34 of the loop 22 is such as to permit the loop 22 to be drawn through the slots 28 and 29 of the cinch clip 24 for urging the cinch clip 24 along the loop 22 towards the anchor bars 20, when a sufficient pulling force is applied to the loop 22, for reducing the effective length of the suture thread 17 between the anchor bars 20 and the cinch clip 24. However, the frictional force exerted by the friction grip of the crimped side walls 26 on the legs 34 of the loop 22 is sufficient for retaining the cinch clip 24 on the loop 22 at a desired position relative to the anchor bars 20 when the effective length of the suture thread 17 between the anchor bars 20 and the cinch clip 24 has been reduced to the desired effective length. This will be described in more detail below.

The maximum external transverse cross-sectional dimension of the cinch clip 24 is between the outer surfaces of the top wall 30 and the bottom wall 32 of the cinch clip 24, and is substantially similar to the external transverse cross-section of the anchor bars 20 so that the cinch clips 24 of the sutures 15 are slideable within the suture bore 14 of the cannula 9. The length of the cinch clip 24 of each suture 15 is the same as the length of each one of the anchor bars 20 of the sutures 15.

In this embodiment of the invention the suture threads 17 of the respective sutures 15 are of nylon material, but may be of any other suitable biocompatible material, for example, polypropylene material or other suitable polymer material. The anchor bars 20 and the cinch clips 24 are of stainless-steel material, but may be of any other suitable biocompatible material, for example, a plastics material or a polymer material, and may, for example, be formed by injection moulding, or any other suitable plastics or polymer forming process. Additionally, the anchor bars 20 and the cinch clips 24 may be of different materials to each other. The total length of the suture thread 17 of each suture 15 from one anchor bar 20 to the other anchor bar 20 through the cinch clip 24, in this particular case is approximately 118 mm, although it will be readily apparent to those skilled in the art that the length of the suture thread will be dependent on the lumen, vessel or organ, the portions of which are to be sutured together.

Returning now to the suturing instrument 1, the diameter of the suture bore 14 of the cannula 9 is sufficient to accommodate the suture thread 17 of each suture 15 along with the anchor bars 20 and the cinch clip 24 of each suture 15 with the anchor bars 20 and the cinch clip 24 of each suture 15 aligned longitudinally with each other in the suture bore 14, and the suture thread 17 extending along the anchor bars 20 and along the cinch clip 24 within the suture bore 14 of the cannula 9. The anchor bars 20 and the cinch clip 24 of each suture 15 may be arranged in the suture bore 14 in any desired order. In this embodiment of the invention the cinch clip 24 of each suture 15 is arranged in longitudinal alignment with the anchor bars 20 of each suture 15 between the respective anchor bars 20, with the opposite ends of the cinch clip 24 abutting adjacent ends of the anchor bars 20.

A pusher element, in this embodiment of the invention an elongated pusher rod 35 of circular external transverse cross-section extending from a proximal end 36 to a distal end 37 is slideably located in the suture bore 14 of the cannula 9 for sequentially discharging the sutures 15 from the suture bore 14 of the cannula 9, and for sequentially discharging the anchor bars 20 and the cinch clip 24 of each suture 15. The distal end 37 of the pusher rod 35 extends into the suture bore 14 of the cannula 9 through a bore 33 extending through the secondary handle 8 and in turn through the proximal end 10 of the cannula 9. The external diameter of the pusher rod 35 is less than the diameter of the suture bore 14 of the cannula 9, so that the pusher rod 35 is slideable in the suture bore 14. The pusher rod 35 is urgeable in the suture bore 14 in a distal direction, namely, in the direction of the arrow B, for selectively and sequentially discharging the sutures 15 from the suture bore 14 of the cannula 9 through the distal piercing tip 12 thereof, and when discharging each suture 15 from the cannula 9, to also discharge the anchor bars 20 and the cinch clip 24 of the suture 15 selectively and sequentially through the distal piercing tip 12 of the cannula 9 in the order in which the anchor bars 20 and the cinch clip 24 of each suture 15 are located in the suture bore 14. The distal end 37 of the pusher rod 35 is configured to engage the proximal most one of the anchor bars 20 or the cinch clip 24, as the case may be, of the proximal most suture 15 in the suture bore 14 of the cannula 9, so that as the pusher rod 35 is selectively and incrementally urged distally through the suture bore 14, the anchor bars 20 and the cinch clips 24 of the respective sutures 15 are sequentially discharged from the suture bore 14 through the distal piercing tip 12 of the cannula 9.

An urging means, in this embodiment of the invention comprising an actuator, which in this case comprises a linear actuator 38 illustrated in block representation in FIG. 1, is configured to incrementally urge the pusher rod 35 through the suture bore 14 for selectively and sequentially discharging the anchor bars 20 and the cinch clip 24 of each suture 15 from the suture bore 14 through the distal piercing tip 12 of the cannula 9 as that suture 15 is being discharged from the cannula 9. The linear actuator 38 may comprise an electrically powered linear motor, or an electrically powered stepper motor, which would transmit drive to the pusher rod 35, through a transmission means, for example, a rack and pinion drive, and may be located internally in or externally on the secondary handle 8, or in any other suitable location.

A controller 39 is responsive to an activation signal to operate the linear actuator 38 to urge the pusher rod 35 distally in the suture bore 14 through a predefined distance.

In this embodiment of the invention the controller 39 comprises a signal processor provided by a programmable microcontroller 39 located in the primary handle 6, which is programmed to activate the linear actuator 38 to urge the pusher rod 35 distally through the predefined distance in response to the activation signal. The length of the predefined distance through which the pusher rod 35 is urged in response to each one of the activation signals is equal to the length of one of the anchor bars 20. Since the length of the anchor bars 20 and the length of the cinch clips 24 are the same, each time the pusher rod 35 is urged distally in the suture bore 14 through the predefined distance, the distal most one of the anchor bars 20 and the cinch clip 24 of the distal most suture adjacent the distal piercing tip 12 of the cannula is urged from the suture bore 14 through the distal piercing tip 12.

The activation signal may be generated or derived from any suitable source, for example, by an electrical switch, a key pad or any other suitable signal generating means. In this embodiment of the invention the activation signal is produced by a human operable electrical switch, which in this case comprises a foot pedal operated electrical switch 40. The foot pedal switch 40 is configured, so that each time it is operated it produces one of the activation signals. The microcontroller 39 is responsive to each activation signal from the foot pedal switch 40 for operating the linear actuator 38 for in turn urging the pusher rod 35 along the suture bore 14 another one of the predefined distances.

A power supply 41 is provided for supplying power to the microcontroller 39 and the linear actuator 38 for powering thereof, and to the foot pedal switch 40.

The outer sleeve 3 of the suturing instrument 1 is of a flexible bendable material in order to accommodate bending and flexing of the outer sleeve 3 during manoeuvring of the suturing instrument 1 through bends and curves in the anatomy of a subject through which the suturing instrument 1 must be passed in order to access the lumen, vessel, organ or cavity in which the suturing is to be carried out. In this embodiment of the invention the material of the outer sleeve 3 comprises a PTFE material, and is formed by extruding the PTFE material. However, it will be understood that the outer sleeve 3 may be of any other suitable material, and in some embodiments of the invention the outer sleeve 3 may comprise a braided cable, for example, a Bowden cable.

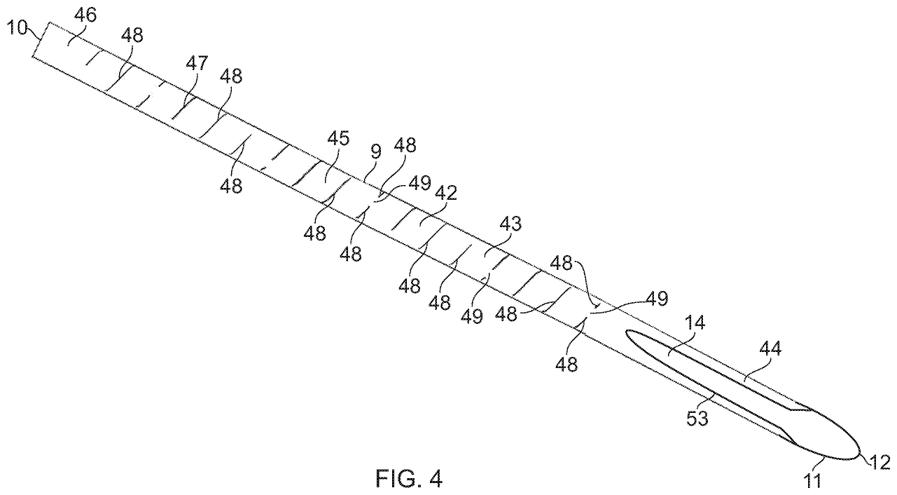

Referring now to FIGS. 4 to 7, the cannula 9 will now be described in detail. The cannula 9 comprises a tubular member 42 of stainless-steel material of constant circular transverse cross-section and having a peripheral wall 43 of constant radial thickness along its length. The outer diameter of the tubular member 42 is approximately 1.27 mm, and the radial thickness of the wall 43 of the tubular member 42 is approximately 0.0762 mm. The tubular member 42 terminates in a distal piercing portion 44 which is substantially rigid for piercing through tissue of the lumen, vessel or organ which is to be sutured. An intermediate portion 45 extends from the distal piercing portion 44 to a proximal end portion 46 of the tubular member 42, which terminates in and defines the proximal end 10 of the cannula 9.

An interrupted helical slot 47 extends through the peripheral wall 43 of the tubular member 42 and extends along the intermediate portion 45 in order to provide the intermediate portion 45 with flexibility and bendability, so that the intermediate portion 45 is sufficiently bendable and flexible for manoeuvring the suturing instrument through bends and curves through which the suturing instrument must be passed in order to gain access to the lumen, vessel or organ in which suturing is to be carried out. In this embodiment of the invention the interrupted helical slot 47 comprises a plurality of helical slot sections 48 interrupted by a plurality of interrupted sections 49. Each interrupted section 49 is located between an adjacent pair of the slot sections 48. Each slot section 48 extends partly around the tubular member 42 an angular distance of approximately 240°, and each interrupted section 49 of the interrupted helical slot extends partly around the tubular member 42 an angular distance of approximately 40° between the adjacent pair of the slot sections 48. The pitch of the interrupted helical slot 47 is approximately 1.2 mm, and the kerf width of the slot of each slot section 48 is approximately 0.025 mm.

The tubular member 42 is sheathed with a fine sleeve 50 of extruded polymer material heat shrunk onto the tubular member 42 over the entire length of the tubular member 42, see FIG. 7. The polymer may be any suitable heat shrinkable polymer material, for example, FEP or PTFE.

A retaining means for releasably retaining the distal most one of the anchor bars 20 or the cinch clip 24, as the case may be, of the distal most one of the sutures 15 in the suture bore 14 comprises an inwardly impressed dimple 51 formed on one side of the peripheral wall 43 in the distal piercing portion 44 of the cannula 9 adjacent the distal piercing tip 12 to form a corresponding inwardly extending retaining projection 52 extending inwardly into the suture bore 14.

A relieving slot 53 extends longitudinally from the distal piercing tip 12 into the distal piercing portion 44 of the cannula 9 to permit outward movement of the dimpled portion of the tubular member 42 adjacent the dimple 51 in order to allow outward movement of the retaining projection 52 for releasing the distal most one of the anchor bars 20 or the cinch clip 24, as the case may be, of the distal most one of the sutures 15, as the pusher rod 35 is being urged distally in the suture bore 14 through the predefined distance for discharging the distal most one of the anchor bars 20 or the cinch clip 24 from the suture bore 14 through the distal piercing tip 12. The inherent resilience of the tubular member 42 is sufficient to return the portion of the tubular member 42 with the dimple 51 to its normal undisturbed state when the distal most one of the anchor bars 20 or the cinch clip 24 has passed the retaining projection 52, so that the next distal most one of the anchor bars 20 or the cinch clip 24 is retained by the retaining projection 52 as it returns to its undisturbed state. The retained next distal most one of the anchor bars 20 or the cinch clip 24 is retained captive by the retaining projection 52, until the pusher rod 35 is urged distally further along the suture bore 14 through the next one of the predefined distances for urging that retained next one of the anchor bars 20 or the cinch clip 24 passed the retaining projection 52 from the suture bore 14.

While the cannula 9 has been described as being of tubular steel, the cannula 9 may be of any other suitable material, and in some embodiments of the invention may be of a plastics or polymer material which would be inherently flexible and bendable, but over a short length, for example, over the length of the distal piercing portion 44 would be substantially rigid, and would be of sufficient rigidity to achieve piercing of the tissue by the distal piercing tip 12 of the cannula 9. Needless to say, while specific linear and angular dimensions of the interrupted helical slot 47 including the slot sections 48 and the interrupted sections 49 thereof have been described, it will be readily understood that the interrupted helical slot 47 may be of any other suitable angular and linear dimensions. Needless to say, in cases where the tubular member 42 of the cannula 9 is of an inherently flexible bendable material, the intermediate portion 45 of the tubular member 42 of the cannula may be provided without an interrupted helical slot.

The advantage of providing an interrupted helical slot 47 in the intermediate portion 45 of the tubular member 42 which is insufficiently flexible and bendable for passing through the various bends and curves in a subject through which the suturing instrument must pass in order to gain access to the lumen, vessel or organ is that the interrupted helical slot 47 while providing the necessary degree of flexibility and bendability does not affect the linear dimensional stability of the tubular member 42, so that the length of the intermediate portion 45 of the tubular member 42 remains constant during bending and flexing thereof, and during urging of the cannula 9 to pierce through a part of the lumen, vessel or organ during suturing thereof.

The pusher rod 35 is likewise of a flexible bendable material, in this embodiment of the invention a stainless-steel material. However, it is envisaged that the pusher rod may comprise an elongated wire of nitinol titanium, and such a pusher rod would have a considerable degree of flexibility and bendability.

To assist in further understanding of the suturing instrument 1, the use of the suturing instrument 1 in suturing two portions of a lumen, vessel or organ together, will now be described with reference to FIGS. 8 to 15. In this example of the use of the suturing instrument 1, the suturing instrument 1 will be described in the carrying out of an endoscopic sleeve gastroplasty procedure, in which a gastric sleeve 59 is formed extending through a stomach 56 of a subject. The gastric sleeve 59 is formed by suturing opposite side walls, namely, a front side wall 54 and a rear side wall 55 of the stomach 56 of the subject together.

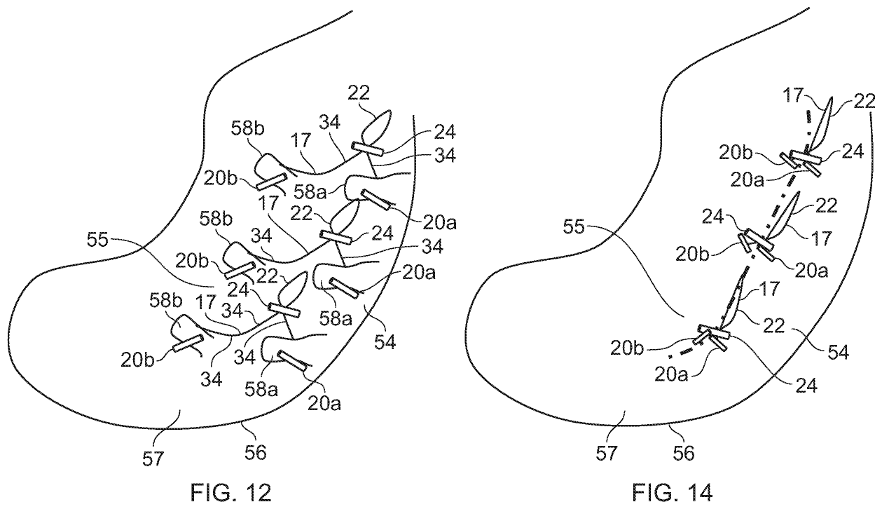

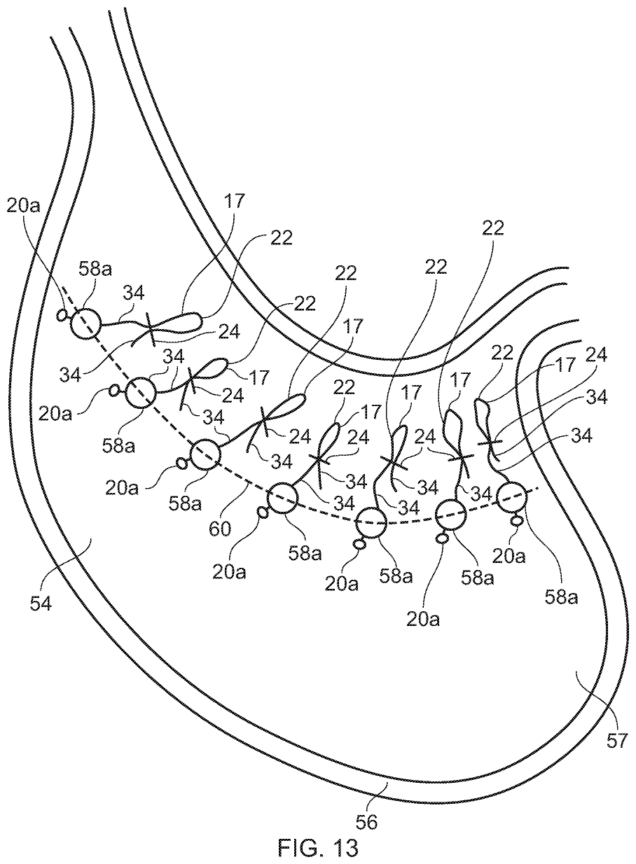

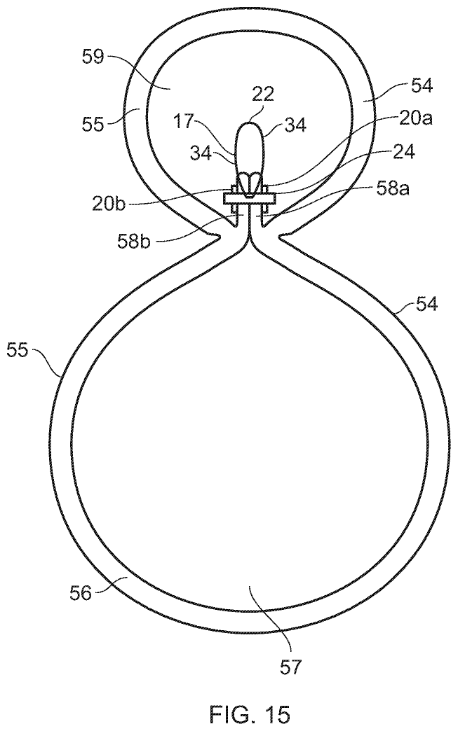

An endoscope (not shown) is initially urged orally through the oesophagus and into the interior 57 of the stomach 56 of a subject. Initially spaced apart parts 58 of the front and rear side walls 54 and 55, along which the side walls are to be sutured together to form the gastric sleeve 59 through the stomach 56, are invaginated to form respective lines 60 of invaginated parts 58a and 58b in pairs thereof on the side walls 54 and 55, respectively. The invaginated parts 58a and 58b of each pair thereof extend from the side walls 54 and 55, respectively, inwardly into the interior 57 of the stomach 56. The invaginated parts 58a of the pairs of the invaginated parts 58a and 58b are illustrated in FIG. 13 spaced apart along the line 60 thereof on the front side wall 54 of the stomach 56. The invaginated parts 58b of the pairs of the invaginated parts 58a and 58b are formed along the line 60 on the rear wall 55 of the stomach 56. One of the invaginated part 58a and one of the invaginated part 58b of one pair thereof are illustrated in FIG. 15 on the front and rear walls 54 and 55, respectively, of the stomach 56. FIG. 12 illustrates three pairs of the invaginated parts 58a and 58b formed on the front and rear walls 54 and 55, respectively, of the stomach 56.

The invaginated parts 58a and 58b of each pair thereof may be formed by any suitable invaginating means, for example, by a multiband ligator which will be well known to those skilled in the art, or by a mechanical grabber. In cases where the invaginated parts 58a and 58b are formed by a multiband ligator, the multiband ligator is passed into the stomach 56 through an instrument bore of the endoscope. As each invaginated part 58a and 58b are formed, a band (not shown) is placed by the multiband ligator around the corresponding one of the invaginated parts 58a and 58b adjacent the root thereof in order to retain the invaginated part 58a or 58b invaginated. The band tightly grips the root of the corresponding invaginated part 58a or 58b.

Once the lines 60 of the invaginated parts 58a and 58b have been formed on the respective opposite side walls 54 and 55 in the interior 57 of the stomach 56 in the pairs thereof, suturing of the stomach to form the gastric sleeve 59 therein commences. The suturing instrument 1 is entered into the stomach 56 through an instrument bore of the endoscope (not shown). During entry of the suturing instrument 1 into the stomach 56, the cannula 9 is in the withdrawn state withdrawn into the outer sleeve 3, so that the distal piercing tip 12 of the cannula 9 is located within the cannula bore 7 of the outer sleeve 3 in order to protect the distal piercing tip 12, and to avoid the distal piercing tip 12 invertedly piercing tissue within the stomach 56.

When the distal end 5 of the outer sleeve 3 is adjacent the first one of the invaginated parts 58a and 58b of the first pair thereof to be sutured, for example, the invaginated part 58a of the distal most pair of the invaginated parts 58a and 58b in the stomach 56, the cannula 9 is urged by the secondary handle 8 from the withdrawn state to the extended state with the distal piercing tip 12 extending distally out of the distal end 5 of the outer sleeve 3. The distal piercing tip 12 of the cannula 9 is then urged through the first one of the invaginated parts 58a of the first pair thereof, see FIG. 8. The foot pedal switch 40 is operated to produce the first one of the activation signals to the microcontroller 39. The microcontroller 39 in turn activates the linear actuator 38 to urge the pusher rod 35 distally through one of the predefined distance, for in turn urging the first and distal most one of the anchor bars 20a of the distal most suture 15 in the suture bore 14 past the retaining projections 52, and in turn, through the distal piercing tip 12, so that the first anchor bar 20a is deposited on the distal side of the first one of the invaginated parts 58a of the first pair thereof, see again FIG. 8.

The cannula 9 is then withdrawn from the first invaginated part 58a of the first pair thereof with the suture thread 17 of the suture 15 extending from the first anchor bar 20a through the first invaginated part 58a of the first pair thereof, see FIG. 9. The foot pedal switch 40 is again operated to produce the next one of the activation signals, which in turn results in the microcontroller 39 operating the linear actuator 38 to again urge the pusher rod 35 distally through another one of the predefined distances for in turn discharging the cinch clip 24 of the distal most suture 15 from the suture bore 14 into the stomach 56, see again FIG. 9.

The distal piercing tip 12 of the cannula 9 is urged through the second invaginated part 58b of the first pair thereof, and the foot pedal switch 40 is again operated to produce the next one of the activation signals. The microcontroller 39 in response to that activation signal again operates the linear actuator 38 to urge the pusher rod 35 distally through a further one of the predefined distances in the suture bore 14 to discharge the second and now the distal most one of the anchor bars 20b of the distal most suture 15 from the suture bore 14, and to deposit the second anchor bar 20b on the distal side of the second invaginated part 58b of the first pair thereof, see FIG. 10. The cannula 9 is withdrawn from the second invaginated part 58b with the suture thread 17 extending from the second anchor bar 20b through the second invaginating part 58b of the first pair thereof, and with the cinch clip 24 located proximally of the first and second invaginated parts 58a and 58b on the loop 22 formed by the legs 34 of the suture thread 17 extending from the first and second anchor bars 20a and 20b, see FIG. 11.

With the suture 15 so inserted in the first and second invaginated parts 58a and 58b of the first pair thereof, the now distal most suture 15 in the suture bore 14 of the cannula 9 is inserted into the next pair of the invaginated parts 58a and 58b in a similar manner to that just described, and so on for the remaining pairs of the invaginated parts 58a and 58b. When sutures 15 have been inserted into all of the pairs of the invaginated parts 58a and 58b, see for example, FIGS. 12 and 13, the suturing instrument 1 is withdrawn through the endoscope (not shown).

A suture tightening instrument is then urged into the stomach 56 through an instrument bore of the endoscope for tightening the sutures 15 to draw the invaginated parts 58a and 58b of each pair thereof together. Each suture 15 is tightened by holding the loop 22 of the suture thread 17, and urging the cinch clip 24 along the legs 34 of the loop 22 towards the anchor bars 20 to reduce the effective length of the suture thread 17 between the anchor bars 20, thereby drawing the invaginated parts 58a and 58b of the corresponding pair thereof together. Any suitable type of suture tightening instrument may be used.

Typically, such a suture tightening instrument would comprise an outer sleeve terminating in a distal abutment end. An operating rod terminating in a distal hook or a distal grabber would be slideable in the outer sleeve for engaging the loop 22 of each suture 15, and for drawing the loop 22 into the outer sleeve until the cinch clip 24 abutted the distal abutment end of the outer sleeve. The loop 22 of the suture 15 would then be drawn further into the outer sleeve of the tightening instrument, for in turn urging the cinch clip 24 along the loop 22 towards the anchor bars 20.

With the hook or grabber extending outwardly from the distal end of the sleeve, the hook or grabber is engaged with the loop 22 of the first of the sutures 15 to be tightened. The hook or grabber is then withdrawn into the sleeve thereby pulling the loop 22 therein until the cinch clip 24 engages the distal abutment end of the sleeve. Once the cinch clip 24 engages the distal abutment face of the sleeve, further pulling of the loop 22 into the bore of the outer sleeve results in the cinch clip 24 being urged along the loop 22 towards the anchor bars 20, thereby progressively reducing the effective length of the suture thread 17 between the anchor bars 20 and the cinch clip 24, for in turn drawing the invaginated parts 58a and 58b of the pair thereof together. Drawing the first and second invaginated parts 58a and 58b together results in the front and rear walls 54 and 55 of the stomach 56 adjacent the corresponding pair of the invaginated parts 58a and 58b being drawn together. As discussed above, the frictional grip of the crimped parts 27 of the side walls 26 of the cinch clips 24 acting on the legs 34 of the loops 22 of the suture thread 17 of the respective sutures 15 is sufficient to retain the cinch clips 24 at the desired position on the loops 22 of the sutures 15, thereby retaining the invaginated parts 58a and 58b of the pair thereof together.

On completion of tightening of the suture 15 of the first pair of the invaginated parts 58a and 58b, the suture 15 on the next adjacent pair of the invaginated parts 58a and 58b is then tightened, and so on until all the sutures 15 have been tightened, thereby drawing the pairs of the invaginated parts 58 together, and in turn drawing the front and rear walls 54 and 55 of the stomach 56 together along the line 60 of the invaginated parts 58 to form the gastric sleeve 59 extending through the stomach 56, see FIG. 15. On completion of tightening of the sutures 15, the suture tightening instrument is then withdrawn through the endoscope.

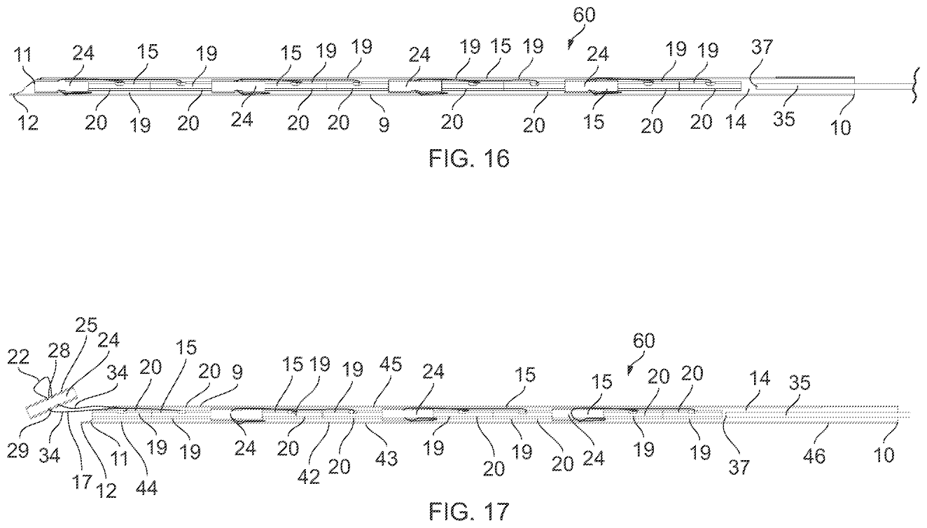

Referring now to FIGS. 16 and 17, there is illustrated a portion of a suturing instrument according to another embodiment of the invention indicated generally by the reference numeral 65. The suturing instrument 65 is substantially similar to the suturing instrument 1 described with reference to FIGS. 1 to 7, and similar components are identified by the same reference numerals. Although in FIGS. 16 and 17 the suturing instrument 65 is illustrated without an outer sleeve, similar to the outer sleeve 3 of the suturing instrument 1. The suturing instrument 65 will be provided with an outer sleeve similar to the outer sleeve 3 of the suturing instrument 1.

The only difference between the suturing instrument 65 and the suturing instrument 1 relates to the manner in which the sutures 15 are located in the suture bore 14 of the cannula 9 of the suturing instrument 65. In this embodiment of the invention each suture 15 is located in the suture bore 14 with the anchor bars 20 located end-to-end and abutting each other, and the cinch clip 24 is located distally of the two anchor bars 20. Otherwise, the suturing instrument 65 is similar to that of the suturing instrument 1.

In use, before each pair of invaginated parts 58 of the lumen, vessel or organ are pierced by the distal piercing tip 12 of the cannula 9 of the suturing instrument 65, the foot pedal switch 40 must be operated to produce the activation signal to the microcontroller 39 in order to operate the linear actuator 38 to urge the pusher rod 35 through one of the predefined distances for discharging the cinch clip 24 of the distal most one of the sutures 15 from the suture bore 14 through the distal piercing tip 12 into the interior of the lumen, vessel or organ. Once the cinch clip 24 has been discharged from the suture bore 14 of the cannula 9, the distal piercing tip 12 of the cannula 9 is urged through the first invaginated part 58a of the first pair thereof. The foot pedal switch 40 is operated for in turn operating the linear actuator 38 to urge the pusher rod 35 through another one of the predefined distances for discharging the distal most one of the anchor bars 20 from the suture bore 14 to the distal side of the pierced invaginated part 58a of the pair thereof. Thereafter, operation of the suturing instrument 65 is similar to the operation of the suturing instrument 1.

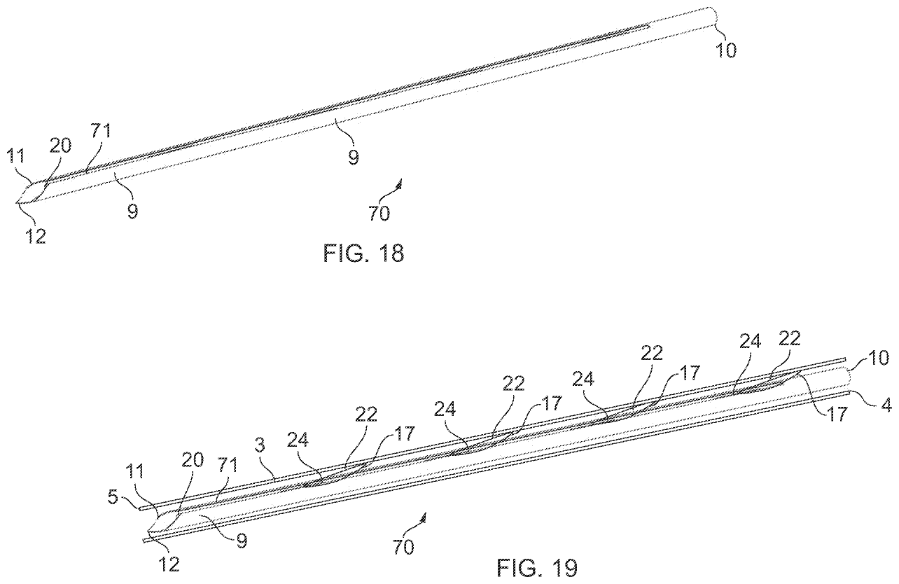

Referring now to FIGS. 18 and 19, there is illustrated a portion of a suturing instrument according to another embodiment of the invention indicated generally by the reference numeral 70. The suturing instrument 70 is substantially similar to the suturing instrument 1, and similar components are identified by the same reference numerals. The main difference between the suturing instrument 70 and the suturing instrument 1 is that in this embodiment of the invention the cannula 9 is not provided with the interrupted helical slot. However, in this embodiment of the invention the tubular member 42 of the cannula 9 is provided with a longitudinally extending slot 71 extending through the peripheral wall 43 of the tubular member 42 from the distal end 11, terminating just short of the proximal end 10 of the cannula 9. The longitudinally extending slot 71 communicates with the suture bore 14 for accommodating the suture threads 17 of the respective sutures 15 therethrough. The majority of the suture threads 17 of the sutures may be located in the slot 71 or may extend through the slot 71, and thus into the cannula bore 7 of the outer sleeve 3. The provision of the slot 71 to accommodate the suture threads 17 therein or therethrough allows the cannula 9 to be provided with a slightly smaller diameter suture bore 14. By reducing the diameter of the suture bore 14 of the cannula 9, the cannula 9 may be provided to be of smaller outer diameter than would otherwise be the case. The diameter of the cannula 9 dictates the diameter of the invaginated parts 58 which must be formed, and therefore by reducing the diameter of the cannula 9, the diameter of the invaginated parts 58 may also be reduced.

A further advantage of the provision of the slot 71, is that it allows the suture threads 17 of the sutures 15 to be located in the cannula bore 7 of the outer sleeve 3, and since the inner and outer diameters of the outer sleeve 3 is less critical than the outer diameter of the cannula 9, the suture threads 17 of the sutures can be stored in the cannula bore 7 of the outer sleeve 3 without having any detrimental effect on the suturing instrument 70.

Otherwise, the suturing instrument 70 and its use are similar to the suturing instrument 1.

The anchor bars 20 and the cinch clips 24 may be arranged in the suture bore 14 of the cannula 9 of the suturing instrument 70 in either of the arrangements described with reference to the cannula 9 of the suturing instrument 1 or in the cannula 9 of the suturing instrument 65. It will of course be appreciated that in some embodiments of the suturing instruments 1, 65 and 70, the anchor bars 20 and the cinch clip 24 of each suture 15 may be arranged with the cinch clip 24 proximal of the two corresponding anchor bars 20.

While the urging means for urging the pusher rod 35 through the predefined distances has been described as comprising a linear actuator, any other suitable actuators may be provided, and such actuators may comprise either linear or rotary motor. When provided as a rotary motor, a transmission system between the rotary motor and the pusher rod would be provided for converting rotational motion of the rotary motor into linear motion of the pusher rod. Such a transmission system may, for example, comprise a rack and pinion drive or a worm and gear drive or both. It will also be appreciated that instead of the actuator being electrically powered, the actuator may be pneumatically powered. Further, it will be appreciated that the linear motor or indeed a rotary motor of the linear actuator may act on the pusher rod through a ratchet system, whereby, the ratchet would sequentially engage spaced apart projections or recesses on the pusher rod for urging the pusher rod through the predefined distances.

It will also be appreciated that any number of sutures may be located in the suture bore of the cannula 9, and the number of sutures which may be located in the suture bore of the cannula would be limited only by the length of the cannula.

While the suturing instruments have been described as comprising sutures of a specific type, it will be readily apparent to those skilled in the art that any other suitable sutures may be used in conjunction with the suturing instruments. It will be appreciated that the sutures may comprise different types of anchor bars and cinch clips to those described. Further, it will be appreciated that while it is desirable, it is not essential that the anchor bars of each suture be of the same length, and furthermore, it will be appreciated that the cinch clips of each suture need not be the same length as the anchor bars thereof.

In cases where the anchor bars of each suture are of different lengths, and the cinch clip of each suture is of a different length to the anchor bars, the microcontroller would be programmed to operate the linear actuator to urge the pusher rod 35 through the appropriate first, second or third predefined distance depending on the lengths of the anchor bars and/or cinch clips for discharging the relevant ones of the anchor bars and/or the cinch clip, as the case may be, and the first, second and third predefined distance through which the pusher rod 35 would be urged by the actuator would correspond to the length of the respective ones of the anchor bars and the cinch clip to be discharged from the suture bore through the distal piercing tip 12 of the cannula.

Needless to say, it is not essential that the anchor bars be of circular transverse cross-section, all that is required is that they be of cross-sectional dimensions suitable for sliding in the suture bore of the cannula. Needless to say, other types of cinch clips may be used in conjunction with the sutures other than a cinch clip of the type illustrated in FIG. 3.

While the cannula and the outer sleeve of the suturing instruments according to the invention have been described as being of circular transverse cross-section, it will be readily apparent to those skilled in the art that the outer sleeve and the cannula of the suturing instruments may be of any suitable transverse cross-section, for example, the cannula and the outer sleeve may be of square, rectangular, triangular, hexagonal, octagonal, or any other suitable transverse cross-section. Further, it is envisaged that the transverse cross-section of the cannula may be different to that of the outer sleeve. Additionally, in some embodiments of the invention it is envisaged that the cannula may be keyed in the outer sleeve in order to prevent rotation of the cannula in the outer sleeve relative to the outer sleeve.

Additionally, it is envisaged that in some embodiments of the invention the suturing instrument may be provided without an outer sleeve.

It is also envisaged that in some embodiments of the invention a sensing means may be provided adjacent the distal end of the cannula which would be capable of identifying and distinguishing between the cinch clip and the anchor elements of each suture, and may also be configured to distinguish between the two anchor elements of each suture. Such a sensing means would produce a signal indicative of the one of the cinch clip and the anchor elements adjacent the distal piercing tip of the cannula and next to be discharged therethrough. The signals from the sensing means would be read by the microcontroller, which would then control the linear actuator to urge the pusher rod distally the appropriate predefined distance for discharging the relevant one of the suture or anchor element adjacent the distal piercing tip of the cannula.

It is also envisaged that the microcontroller 39 may be pre-programmed to operate the linear actuator through three predefined distances, for example, a first predefined distance to discharge one of the anchor bars of each suture from the suture bore of the cannula, a second predefined distance to discharge the other one of the anchor bars of each suture from the suture bore of the cannula, and a third predefined distance to discharge the cinch clip of each suture from the suture bore of the cannula. It is envisaged that the sequence of the first, second and third predefined distances through which the linear actuator 38 would be operated would be selectable, and would be selectable to match the sequence in which the anchor bars and the cinch clips of the respective sutures are located in the suture bore of the cannula. It is also envisaged that the lengths of the first, second and third predefined distances would be such as to match the lengths of the respective anchor elements and the cinch clip, and would be selectable.

While the suturing instrument has been described for use in an endoscopic sleeve gastroplasty procedure, it will be readily apparent to those skilled in the art that the suturing instruments according to the invention may be used for suturing any two portions of a lumen, vessel or organ together either internally in or externally of the lumen, vessel or organ.

It will also be appreciated that while the suturing instruments have been described for suturing the portions of the lumen, vessel or organ together by forming invaginated parts of the lumen, vessel or organ adjacent the areas which are to be sutured together, while this is an advantageous way of suturing portions of a lumen, vessel or organ together, other suitable methods may be used. For example, in some embodiments of the invention it is envisaged that the suturing instrument may be operated within the lumen, vessel or organ, and the distal piercing tip 12 of the cannula 9 would be urged through one of the portions of the wall of the lumen, vessel or organ to be sutured from the interior thereof, and one of the anchor bars of the suture would be deposited on the exterior side of the wall of that portion of the lumen, vessel or organ. The distal piercing tip of the cannula would then be operated to pierce through the other portion of the wall of the lumen, vessel or organ to be sutured and would deposit the other one of the anchor bars of the suture on the exterior side of the wall of that portion of the wall of the lumen, vessel or organ. Further sutures would be inserted sequentially along the portions of the lumen, vessel or organ at spaced apart intervals along the portions thereof in a similar manner. The sutures would then be tightened, thereby pulling the two portions of the wall or walls of the lumen, vessel or organ together. Needless to say, such a suturing operation could be carried out externally of the lumen, vessel or organ whereby the suturing instrument would be operated to pierce through the wall of the lumen, vessel or organ from the exterior to the interior thereof.

It is also envisaged that as well as the suturing instrument being entered into the lumen, vessel or organ to be sutured through the instrument bore of an endoscope, the suturing instrument may be entered into a lumen, vessel or organ or indeed a cavity in which suturing is to be carried out through the instrument bore of any other suitable access device, for example, a laparoscope, colonoscope, or any other suitable access devices.

While the parts to be invaginated have been described as being invaginated by a multiband ligator, any other suitable invaginating means for forming invaginated parts may be used, for example, a grabber or a grasper, which would grab or grasp a part of the portion of the lumen, vessel or organ to be sutured and would pull the part inwardly, or outwardly as the case may be from the wall of the subject, and while the part is grabbed or grasped, the cannula would be operated to pierce through the grabbed or grasped part, and the distal most one of the anchor bars of the distal most suture in the suture bore would be dispensed on the distal side of the grabbed or grasped part. Once the cannula has been withdrawn from the grabbed or grasped part, the grabber or grasper would release the invaginated part.

1. A suturing instrument comprising:
an elongated cannula extending from a proximal end to a distal end and having a suture bore extending longitudinally therethrough for accommodating a plurality of sutures therein, the cannula terminating at its distal end in a distal piercing tip,
the sutures being of the type comprising:
a suture thread terminating at its opposite ends in respective
anchor elements,
the suture thread being configured to form
a loop extending from one of the anchor elements to the other one of the anchor elements, and
a cinch clip located on the loop of the suture thread and being urgeable along the loop towards the anchor elements for progressively reducing the effective length of the suture thread between the anchor elements,
the suture bore of the cannula being configured for accommodating at least a part of the sutures
therein with at least the anchor elements of the sutures being located in the suture bore, and
a pusher element for selectively and sequentially discharging the suture from the suture bore through the distal piercing tip of the cannula, and for selectively and sequentially discharging the anchor elements of each suture through the distal piercing tip of the cannula.

2. A suturing instrument as claimed in claim 1 in which the suture bore of the cannula is configured for accommodating the cinch clips of the respective sutures.

3. A suturing instrument as claimed in claim 2 in which the pusher element is configured to selectively and sequentially discharge the cinch clip and the anchor elements of each suture, from the suture bore through the distal piercing tip of the cannula.

4. A suturing instrument as claimed in claim 1 in which the pusher element extends into the suture bore from the proximal end of the cannula and is slideable in the suture bore.

5. A suturing instrument as claimed in claim 2 in which an urging means is provided for urging the pusher element distally in the suture bore for selectively and sequentially discharging the anchor elements from the suture bore.

6. A suturing instrument as claimed in claim 5 in which the urging means is configured to urge the pusher element distally selectively and sequentially through a plurality of respective predefined distances for selectively and sequentially discharging the ones of the anchor elements and the cinch clip of each suture in the suture bore of the cannula through the distal piercing tip of the cannula.

7. A suturing instrument as claimed in claim 5 in which the urging means is configured to urge the pusher element distally in the suture bore of the cannula through a first predefined distance for discharging each one of the anchor elements of each one of the sutures through the suture bore.

8. A suturing instrument as claimed in claim 7 in which the first predefined distance is approximately equal to the length of each anchor element of each suture.

9. A suturing instrument as claimed in claim 5 in which the urging means is configured to urge the pusher element distally in the suture bore of the cannula through a second predefined distance for discharging the cinch clip of each one of the sutures from the suture bore.

10. A suturing instrument as claimed in claim 9 in which the second predefined distance is of length approximately equal to the length of the cinch clip of each suture.

11. A suturing instrument as claimed in claim 9 in which the first and second predefined distances are of equal length.

12. A suturing instrument as claimed in claim 5 in which the urging means comprises a linear motor.

13. A suturing instrument as claimed in claim 5 in which the urging means comprises a rotary stepper motor.

14. A suturing instrument as claimed in claim 5 in which a transmission means is provided for transmitting drive from the urging means to the pusher element.

15. A suturing instrument as claimed in claim 5 in which a controller is provided for controlling the operation of the urging means, the controller being responsive to an activation signal for operating the urging means to urge the pusher element distally in the suture bore through the one of the respective predefined distances corresponding to the one of the anchor elements and the cinch clip adjacent the distal piercing tip of the cannula.

16. A suturing instrument as claimed in claim 15 in which the activation signal is derived from a human operable switch.

17. A suturing instrument as claimed in claim 1 in which a retaining means is provided for releasably retaining the sutures in the suture bore.

18. A suturing instrument as claimed in claim 1 in which the cannula terminates in a substantially rigid distal piercing portion extending proximally from the distal piercing tip.

19. A suturing instrument as claimed in claim 18 in which the cannula comprises an intermediate portion extending from the distal piercing portion to a proximal portion adjacent the proximal end of the cannula, the intermediate portion of the cannula being configured to be flexible and bendable.

20. A suturing instrument as claimed in claim 19 in which an interrupted helical slot extends through the peripheral wall of the intermediate portion of the cannula, and the interrupted helical slot extends along the intermediate portion thereof.

21. A suturing instrument as claimed in claim 1 in which the suturing instrument comprises an outer sleeve extending from a proximal end to a distal end and having a cannula accommodating bore extending therethrough and configured for slideably accommodating the cannula therein.

22. A suturing instrument as claimed in claim 1 in which a plurality of the sutures are located sequentially in the suture bore of the cannula.
